(19) United States
(12) Patent Application Publication
(10) Pub. No.: US 2006/0121125 A1
(43) Pub. Date: Jun. 8, 2006

(54) Formulation which can be administered gastrointestinally, containing green tea extract and an no donor

(76) Inventor: Heinz Schneider, Cordast (CH)

Correspondence Address: CONNOLLY BOVE LODGE & HUTZ, LLP, P O BOX 2207, WILMINGTON, DE, 19899, US

(21) Appl. No.: 10/538,223

(22) PCT Filed: Nov. 13, 2003
(86) PCT No.: PCT/EP03/12675

§ 371 Date: Jun. 29, 2005

(30) Foreign Application Priority Data
Dec. 9, 2002 (DE) 102 57 360.3

(51) Int. Cl. A61K 36/82; A61K 33/00; A61K 31/198; A61K 31/537

(52) U.S. Cl. 424/608; 424/729; 514/508; 514/565; 514/237.5

(57) ABSTRACT

A formulation which can be administered gastrointestinally and comprises green tea extract and at least one NO donor which is a substrate of NO synthetase, and/or a precursor thereof is described. The formulation is administered before surgical procedures, in order to avert or reduce the risk of postoperative complications.

The present invention relates to a medicinal or nutrient formulation which can be administered gastrointestinally and to the use thereof for averting or reducing the risk of postoperative complications.

Serious surgical procedure must frequently be expected to be associated with postoperative complications. The latter may have various symptoms, but can often be attributed to the circumstance that the tissue affected by the operation has an inadequate blood supply. So-called ischaemia/reperfusion phenomena may lead to life-threatening conditions in surgical patients.

To date, such complications have mainly been treated only when they have already occurred.

However, approaches to reducing the risk of the occurrence of or the effects of postoperative complications by preparing a patient before an operation by administration of selected formulations have already been described.

Thus, U.S. Pat. No. 5,656,608 describes a method for the treatment of endotoxaemia, comprising the administration of an effective amount of a selected amino acid such as glycine, alanine or serine. The supplementation takes place at least three days before the operation.

U.S. Pat. No. 5,731,290 discloses a method for improving the immune response or the resistance to infections after surgical procedures, entailing preoperative administration of a dietary supplement. The latter has an amount, stimulating the immune system, of omega-3 fatty acids (highly unsaturated fatty acids) combined with L-arginine, L-ornithine or their precursors. Administration of the supplement takes place over a period of at least three days before the operation.

WO-A 96/25 861 describes the use of glycine or the glycine precursors alanine and serine for the preparation of a medicament or of a nutritional formulation for diminishing the content of tumour necrosis factor (TNF) in patients in whom homeostasis or local inflammations are present. This publication mentions the possiblity of supplementation over a period of at least three days before an operation.

WO-A 99/62 508 discloses the use of glycine for the manufacture of a medicament for the treatment of haemorrhagic shock. The possibility of preoperative administration is described.

U.S. Pat. No. 5,902,829 discloses a method for influencing the microcirculation in patients, in which L-arginine or a precursor thereof or another NO donor which is a substrate of NO synthetase or a precursor thereof are administered preoperatively. The supplementation takes place over a period of at least one day before the operation.

U.S. Pat. No. 6,013,273 describes a method for the treatment of endotoxic shock which comprises administering an effective amount of choline. The administration takes place over a period of at least one day before the operation and ordinarily from one to six days before the operation.

It is common to all these treatments that they must be performed for at least one day, ordinarily a plurality of days, before an operation. For patients with an acute need for surgery on in emergency cases (e.g. injuries), the time available is frequently insufficient to achieve a satisfactory result with known methods.

Starting from this state of the art, the present invention provides a formulation with which support of a patient shortly before an operation is possible in order to reduce the risk of the occurrence of or the effects of postoperative complications.

The present invention further provides a method for the prophylaxis of postoperative complications which is aimed in particular at the support of emergency patients.

The formulation according to the invention or the method according to the invention can be used in cases in which only a short period, for example a few hours, is available; they can, of course, also be used in longer periods before an operation.

The formulation according to the invention comprises green tea extract as one ingredient. It has been known for a long time that green tea extract can be employed in the treatment of certain diseases and. that this extract has antibacterial properties. Thus, JP-A 73/30 599 describes the use of polyphenols from green tea for cancer therapy.

Neuroscience Letters 287 (2000), 191-4, discloses the protective effect of (−)-epigallocatechin gallate, the main constituent of the green tea polyphenols, against ischaemia/reperfusion damage in the brain.

Am. J. Physiol. Gastrointest. Liver Physiol., 2002, 283(4): G957-64, discloses that ischaemia/reperfusion damage to the liver can be prevented by administration of green tea extract.

JP-A 10/248 538 describes a composition for increasing the activity of the alpha waves of the brain comprising green tea extract, amino acids, vitamins and sugars.

EP-A 1 174 143 discloses employing green tea extracts for use in the treatment of renal failure induced by cyclosphorins or ascomycins. This publication also discloses compositions which comprise green tea extract and selected amino acids such as glycine, L-alanine, L-serine or L-arginine.

“Chemistry and Applications of Green Tea” (T. Yamamoto et al.) describes the beneficial effect of green tea extract on the intestinal flora and its positive influence on the immune system.

In addition, F. Yang et al. report, in J. Nutr., 1998, 128: 2334-2340 “Green Tea polyphenols block endotoxin-induced TNF-production and lethality in a murine model”, that polyphenols from green tea have anti-inflammatory and anticancer properties and that administration of green tea polyphenols reduces the serum content of TNF-alpha.

It has now been found, surprisingly, that support of surgical patients is possible by administering a composition comprising green tea extract and selected other components such as selected amino acids or amino acid derivatives, and that the risk of postoperative complications can be distinctly decreased, and the effects of such complications can be considerably reduced, thereby.

The present invention relates to the invention the use of a composition comprising
a) green tea extract and
b) at least one NO donor which is a substrate of NO synthetase and/or at least one precursor of this NO donor,
   for the preparation of a formulation for gastrointestinal administration before surgical procedures, in order to reduce the risk of postoperative complications or to avert such a risk.
The present invention further relates to a method for the support of surgical patients against the risk of postoperative complications comprising gastrointestinal administration of a composition comprising
a) green tea extract and
b) at least one NO donor which is a substrate of NO synthetase, and/or at least one precursor of this NO donor,
   before a surgical procedure.

The term formulation which can be administered gastrointestinally means within this description a formulation which can be administered anywhere in the gastrointestinal tract. Preferred adminstration forms are oral intake or administration by stomach tube or intestinal tube.

A surgical procedure means within this description any surgical procedures, but preferably elective surgical procedures or emergency surgical procedures.

Examples of elective surgical procedures are gastrointestinal procedures, heart surgery, nose and throat surgery, abdominal procedures, including minimally invasive abdominal procedures, vascular and joint surgery or transplantations.

Examples of emergency surgical procedures are traumatology procedures or procedures for clearing up a septic focus.

The formulation according to the invention is preferably administered a few days before a surgical procedure, for example between the third preoperative day and the operation. The administration preferably takes place within twenty-four hours before the operation, in particular less than twelve hours, particularly preferably less than six hours, and very especially less than three hours, before the operation.

The formulation according to the invention may, besides components a) and b), comprise as optional component c) glycine, a glycine precursor, preferably in the form of a di- or tripeptide, of the physiologically tolerated salts thereof or combinations thereof.

Besides component a), b) and optionally c), the formulation according to the invention may also comprise further components, for example flavourings or aromas and fillers (e.g. organoleptics) and colours.

The described formulation is administered gastrointestinally to the patient, preferably orally or by stomach tube or intestinal tube.

Formulations which can be administered orally may be employed for example in the form of pills, tablets, film-coated tablets, effervescent tablets (for preparing aqueous solutions, emulsions or suspensions), sugar-coated tablets, granules, hard and soft gelatin capsules, aqueous, alcoholic or oily solutions, syrups, emulsions or suspensions.

On administration by stomach tube or intestinal tube, the formulations are ordinarily administered in the form of aqueous, alcoholic or oily solutions, syrups, emulsions or suspensions.

The formulations can be produced by the known standard processes.

For this purpose, components a), b) and optionally c) are converted together with one or more solid or liquid pharmaceutical carriers and/or additives or excipients, and energy carriers such as carbohydrates, fats and/or proteins and, if desired, in combination with other nutrients having prophylactic benefits into a suitable administration form or dosage form, which can then be used in the manner according to the invention.

The formulations comprise a prophylactically beneficial dose of components a), b) and optionally c), which normally accounts for 0.5 to 90 percent by weight of the formulation.

It is possible for the production of for example pills, tablets, sugar-coated tablets and hard gelatin capsules to use lactose, starch, for example maize starch, or starch derivatives, talc, stearic acid or salts thereof. Carriers for soft gelatin capsules are, for example, fats, waxes, semisolid and liquid polyols, natural or hardened oils. Examples of suitable carriers for producing solutions or emulsions or suspensions or syrups are water, surgical saline, alcohols such as ethanol, glycerol, polyols, sucrose, invert sugar, glucose, mannitol, vegetable oils.

Components a), b) and optionally c) may also be lyophilized, and the resulting lyophilizates be used for example for producing products for infusion. Suitable carriers for microcapsules, implants or rods are, for example, copolymers of glycolic acid and lactic acid.

The formulations may, besides components a), b) and optionally c), also comprise carriers and other conventional additives, for example fillers, disintegrants, binders, lubricants, wetting agents, stabilizers, emulsifiers, dispersants. preservatives, sweeteners, colorants, flavourings or aromatizing agents, thickeners, diluents, buffer substances, also solvents or solubilizers or means for achieving a depot effect, salts to alter the osmotic pressure, coating agents, vitamins or antioxidants.

The formulations are particularly preferably employed in the form of aqueous emulsions, suspensions or, in particular, solutions.

The dosage of components a), b) and optionally c) to be administered depends on the individual case and should be adapted as usual to the individual circumstances for optimal benefit. Thus, it depends on the nature and extent of the complications to be expected and on the sex, age, weight and individual response of the person to be treated, and on the bioavailability of the prophylactically administered components.

Typical contents of component a) vary in the range from 0.01 to 2.0 g, preferably in the range from 0.1 to 1.5 g, in each case based on 1000 ml of a formulation. The weight data are based on the polyphenols present in the green tea extract.

Typical contents of component b) vary in the range from 1 to 150 g, preferably in the range from 0.1 to 30 g, in each case based on 1000 ml of the formulation.

Typical contents of component c) vary in the range from 00.1 to 80 g, preferably in the range from 0.1 to 30 g, in each case based on 1000 ml of the formulation.

The remainder of the formulation preferably consists of water.

The formulations normally comprise an energy content of less than 800 kcal, preferably 100 to 500 kcal, in each case based on 1000 ml.

High-energy additions such as carbohydrates and/or fats and/or proteins are preferably not present. However, to improve the taste it is possible for small amounts, for example 1 to 120 g/1000 ml, of carbohydrates and/or fats and/or proteins to be present.

Typical amounts of component a) administered during the complete treatment period vary in the range from 5 to 2000 mg, preferably 10 to 1600 mg, based on the polyphenol content of component a).

Typical amounts of component b) administered during the complete treatment period vary in the range from 0.1 to 50 g, based on the individual free amino acids.

Typical amounts of component c) administered during the complete treatment period vary in the range from 0.1 to 40 g, based on the free amino acid.

The formulation can be administered in a single dose or, especially on administration of larger amounts, be divided into a plurality of, for example two, three or four, single doses. It may be necessary where appropriate, depending on the individual behaviour, to deviate upwards or downwards from the stated dose.

Any commercially available extracts can be used as green tea extract. These may be water- or oil-soluble green tea extracts.

The green tea extract employed according to the invention preferably comprises the constituents normally present in green tea. Examples thereof are amino acids, polyphenols, vitamins, saccharides, minerals and caffeine.

The preferred amino acids present in green tea extract include theanine.

Preferred polyphenols present in green tea extract include catechin derivatives, that is to say (−)-epigallocatechin gallate (EGCg), (−)-epigallicatechin (EGG), (−)-epicatechin gallate (ECg), (+)-gallocatechin (GC), (−)-epicatechin (EC) and (+)-catechin (C), and mixtures of two or more of these components.

Commercially available products can be employed as basis for green tea extracts to be employed according to the invention. Examples thereof are the products Sunphenon 100S, Sunphenon DCF-1, Sunphenon BG, Sunphenon LA-50, and Sunkatol (all obtainable from Taiyo Kagaku Corp., Japan).

The green tea extract employed according to the invention can be obtained by treating leaves of green tea with hot water at, for example, 80 to 95° C. In such cases, five to ten parts by weight of water are ordinarily employed for one part by weight of tea leaves. The extraction normally lasts ten to 40 minutes. Extraction is followed by filtration, and the filtrate is concentrated in vacuo, for example to one quarter of the original volume, or until all the water has evaporated, so that a powder results. This filtrate or powder can be employed directly for producing the formulation according to the invention.

It is also possible in place of this to employ a concentrate. The latter can be obtained by multiple extraction from the filtrate described above using a suitable extractant, for example ethyl acetate.

For this purpose, the filtrate described above or the concentrate is extracted by shaking, for example several times, with an approximately identical volume of ethyl acetate, in order to extract the active constituents of green tea. The combined extracts are concentrated to constant weight in vacuo. The resulting residue can be employed as component a).

For use in the formulation according to the invention, green tea extract is employed in dried or liquid form, for example in the form of a powder, of an oil or of an aqueous solution. Aqueous solutions are preferably employed.

The green tea extract employed preferably includes theanine and polyphenols derived from catechin derivatives, in particular from the catechin derivatives described above as preferred.

A wide variety of compounds can be employed as NO donors which are a substrate of NO synthetase and/or as precursors thereof.

Examples thereof are amino acids having activity as substrate of NO synthetase, especially arginine and glutamine, precursors of these amino acids, their physiologically tolerated salts or combinations of these compounds.

Likewise preferably employed as NO donors which are substrates of NO synthetase are trinitroglycerin, isosorbite dinitrate, nitroprussite, aminoguanidine, spermine-NO, spermidine-NO and SIN 1 (3-morpholinosydnone imines).

The term precursors of amino acids mean compounds which contain the relevant amino acid or precursor thereof and which lead to release of the relevant amino acid through metabolic activities.

Examples of amino acid precursors are derivatives of the amino acid, such as esters, amides, N-alkylated or N-acylated amino acid, salts or keto precursors thereof, and short-chain peptides such as di- to decapeptides, preferably tripetides, and very particularly preferably dipeptides, which contain the relevant amino acid. Examples of tripeptides are X-AA-X′, X-X′-AA and X-AA-AA, where X and X′ represent naturally occurring amino acids, and AA represents the relevant amino acid.

Amino acid derivatives are preferably employed in the form of tri- and, in particular, dipeptides.

Examples of preferred derivatives of arginine are the dipeptides Ala-Arg, Arg-Ala, Arg-Gly and Gly-Arg.

Examples of preferred derivatives of glutamine are the dipeptides Ala-Gln and Gly-Gln.

Examples of preferred derivatives of glycine are the dipepptides Ala-Gly, Gly-Ala and Gly-Gly.

Examples of physiologically tolerated salts are phosphates, citrates, acetates, malates, tartrates, fumarates, lactates and hydrates.

Compositions which are preferably employed are those in which component b) is arginine or an arginine precursor in the form of a di- or tripeptide.

The invention also relates to the use of a medicinal or nutrient formulation which can be administered gastrointestinally and comprises
a) green tea extract and
b) at least one NO donor which is a substrate of NO synthetase, and/or at least one precursor of this NO donor, in particular a compound selected from the group consisting of arginine, glutamine, precursors of these amino acids, trinitroglycerin, isosorbite dinitrate, nitroprussite, aminoguanidine, spermine-NO, spermidine-NO and SIN 1 (3-morpholinosydnone imine), the physiologically tolerated salts or combinations thereof
   to avert or reduce the risk of postoperative complications after surgical procedures.

The following example illustrates the invention without limiting it.

Ischaemia-Reperfusion Modell

Every surgical procedure involves reversible ischaemias of organs and tissues caused by temporary clamping of blood vessels. Such an ischaemia situation can be simulated in a low-flow, reflow liver perfusion model in rats.

In this rat model, the liver is perfused at a low flow rate in order to generate a local oxygen deficiency, i.e. anoxia, due to a reduced oxygen supply in the pericentral liver region. Subsequent reperfusion of the liver at normal flow rates introduces oxygen again into the previously anoxic liver regions, then leading to an oxygen-dependent formation of free radicals and corresponding reperfusion damage. The cell death caused thereby of many cells in the pericentral liver region releases cell-bound enzymes, e.g. lactate hydrogenase and transaminases, and they can be detected and quantified in the perfusate.

Sprague-Dawley rats weighing 200-250 g are fasted overnight. A first portion of the animals receives a combination of green tea extract and L-arginine administered into the stomach (gavage). An identical volume of water is administered to a second group (control). The rats are subsequently anaesthetized with phenobarbital, and the abdomen is opened. A partial ischaemia of the liver is performed, clamping the artery and portal vein for the three upper lobes of the liver (about 70% of the total liver mass) for one hour. The liver is then reperfused and the surgical wound is closed.

An increase in serum transaminases is measured after 1.5; 3; 7 and 24 hours.

In the control group, the increase in transaminases after 7 hours is more than 50-fold, whereas a significantly smaller increase in these enzyme activities is observed under the conditions of administration of the combination of green tea extract and L-arginine.

This effect demonstrates the action of the combination of green tea extract and L-arginine in preventing ischaemia-reperfusion damage.

1-2. (canceled)

3. The method of claim 10, wherein the surgical procedure is an elective surgical procedure or an emergency surgical procedure.

4. The method according to claim 3. wherein the elective surgical procedure is a gastrointestinal procedure, heart surgery, nose and throat surgery, an abdominal procedure, vascular and/or joint surgery or transplantations.

5. The method of claim 3. wherein emergency surgical procedure is trauma surgery or procedures for clearing up a septic focus.

6. The method of claim 10, wherein the green tea extract includes theanine and polyphenols derived from catechin derivatives.

7. The method of claim 6, wherein the catechin derivatives are selected from the group consisting of (−)-epigallocatechin gallate (EGCg), (−)-epigallicatechin (EGG), (−)-epicatechin gallate (ECg), (+)-gallocatechin (GC), (−)-epicatechin (EC), (+)-catechin (C) and combinations of two or more constituents thereof.

8. The method of claim 10, wherein component b) is arginine or an arginine precursor in the form of a di- or tripeptide.

9. The method of claim 10,wherein said composition further comprises as component c) glycine, a glycine precursor in the form of a di- or tripeptide, or the physiologically tolerated salts thereof or combinations thereof.

10. A method for the support of surgical patients against the risk of postoperative complications comprising the step of gastrointestinally administering a composition comprising
a) green tea extract and
b) at least one NO donor which is a substrate of NO synthetase, and/or one precursor of this NO donor,
before a surgical procedure.

11. The method according to claim 10, wherein the NO donor which is a substrate of NO synthase, or the precursor of this NO donor, is selected from the group consisting of arginine, glutamine, precursors of these amino acids, trinitroglycerin, isosorbite dinitrate, nitroprussite, aminoguanidine, spermine-NO, spermidine-NO and SIN 1 (3-morpholinosydnone imines), or the physiologically tolerated salts or combinations thereof.

12. The method according to claim 10, wherein the administration of the composition comprising components a) and b) takes place less than twenty-four hours, before the surgical procedure.

13-14. (canceled)

15. The method of claim 10, wherein said support of surgical patients against the risk of postoperative complications comprises averting or reducing the risk of postoperative complications.

16. A formulation for gastrointestinal administration before surgical procedures to reduce the risk of postoperative complications or to avert such a risk comprising a composition comprising
a) green tea extract and
b) at least one NO donor which is a substrate of NO synthetase, and/or one
precursor of this NO donor.

17. The formulation of claim 16, wherein the NO donor which is a substrate of NO synthase, or the precursor of this NO donor, is selected from the group consisting of arginine, glutamine, precursors of these amino acids, trinitroglycerin, isosorbite dinitrate, nitroprussite, aminoguanidine, spermine-NO, spermidine-NO and SIN 1 (3-morpholinosydnone imines), or the physiologically tolerated salts or combinations thereof.

18. The formulation of claim 16, wherein the green tea extract includes theanine and polyphenols derived from catechin derivatives.

19. The formulation of claim 18, wherein the catechin derivatives are selected from the group consisting of (−)-epigallocatechin gallate (EGCg), (−)-epigallicatechin (EGG), (−)-epicatechin gallate (ECg), (+)-gallocatechin (GC), (−)-epicatechin (EC), (+)-catechin (C) and combinations of two or more constituents thereof.

20. The formulation of claim 16, wherein component b) is arginine or an arginine precursor in the form of a di- or tripeptide.

21. The formulation of claim 16, wherein said composition further comprises as component c) glycine, a glycine precursor in the form of a di- or tripeptide, or the physiologically tolerated salts thereof or combinations thereof.
